(19) United States
(12) Patent Application Publication
(10) Pub. No.: US 2006/0205651 A1
(43) Pub. Date: Sep. 14, 2006

(54) Method of treating cancer

(75) Inventor: Judith Harmey, Dublin 2 (IE)

Correspondence Address: WOLF GREENFIELD & SACKS, PC, FEDERAL RESERVE PLAZA, 600 ATLANTIC AVENUE, BOSTON, MA, 02210-2206, US

(73) Assignee: ROYAL COLLEGE OF SURGEONS IN IRELAND, Dublin 2 (IE)

(21) Appl. No.: 11/360,921

(22) Filed: Feb. 23, 2006

Related U.S. Application Data
(60) Provisional application No. 60/656,641, filed on Feb. 25, 2005.

(51) Int. Cl. A61K 38/17; A61K 38/22

(52) U.S. Cl. 514/12

(57) ABSTRACT

The invention relates to a method of treating cancer in an individual in need thereof including inhibiting tumour growth and metastasis. The invention also relates to a method of suppressing or preventing formation of metastases, or inhibiting the growth of metastases, particularly bone metastases, from primary tumours.

### RELATED APPLICATION

This application claims the benefit under 35 U.S.C. § 119(e) of U.S. provisional application 60/656,641, filed Feb. 25, 2005, the entire disclosure of which is incorporated herein by reference.

### FIELD OF THE INVENTION

The invention relates to a method of treating cancer in an individual in need thereof. In particular, the invention relates to a method of inhibiting tumour growth and metastasis. The invention also relates to a method of suppressing or preventing formation of metastases, particularly bone metastases, from primary tumours. The invention also relates to a method of inhibiting the growth of metastases.

### BACKGROUND OF THE INVENTION

The insulin-like growth factors (IGFs) play an important role in normal growth and development. Evidence suggests they may also regulate the growth of several cancer cell types. This regulation is mediated by interactions between the receptors of insulin-like growth factors (IGF-Rs or IGFRs) and ligands (IGFs). There is now evidence to suggest that these interactions are also influenced by extracellular IGF-binding proteins (IGFBPs). Six different IGFBPs have been cloned.

Insulin-like growth factor-binding proteins (IGFBPs) both stimulate and inhibit IGF activity. IGFBPs can affect cell function in an IGF-dependent or -independent manner. The proteolytic cleavage of IGFBPs by various proteases decreases dramatically their affinity for their ligands and therefore enhances the bioavailability of IGFs. Some species may act to inhibit the mitogenic effects of the IGFs. Antimitogenic effects of IGFBP-4 have been demonstrated in different cellular systems, such as human fibroblasts, osteoblasts, neuronal cells, and in human prostate cancer cells. Notably, both IGF-dependent and -independent mechanisms have been suggested for the antimitogenic effects of IGFBP-4.

Insulin-like growth factor 1 (IGF-1 or IGF I) has mitogenic properties for breast cancer cell lines and has been proposed to be an important factor in breast carcinogenesis. In breast cancer there is evidence that IGF-1 promotes breast cancer and has a role in the progression of the disease. Many breast cancer cells produce IGFs and possess the appropriate IGF receptors, and therefore the IGFs can act in an autocrine fashion (Rasmussen et al., 1998). Plasma levels of IGF-1 are elevated in breast cancer patients (Peyrat J P et al., 1993). In vitro, IGFR-1 is expressed by many breast cancer cell lines and IGF-1 is mitogenic.

There is evidence that the IGF system also plays a role in invasion and metastasis. IGF-1 stimulates tumour cell invasion in part by inducing urokinase plasminogen activator. There is evidence that the IGF-1R (IGF-1 receptor) plays a role in angiogenesis and lymphangiogenesis through the induction of vascular endothelial growth factors (VEGF 165 and VEGF121). Thus, IGF-1R affords breast cancer cells many opportunities to become invasive and eventually metastatic (Kucab J. E. et al., 2003). In one study, elevated IGFBP4 was associated with breast cancer. These authors suggest that the bioavailability of IGF-1 as mediated by its binding proteins may participate in both breast carcinogenesis and selection of more aggressive breast carcinomas (Ng E. H. et al., 1998). Another study also found that IGFBP-4 expression in breast tumours correlated with poor prognosis (Yee D et al., 1994).

In contrast, another study found no correlations between ER (estrogen receptor, a marker of good prognosis and less aggressive tumour) status, or parameters related to the hormonal status, and IGF-I or IGF binding proteins expression. No significant differences in IGF-I concentration and IGF-BP expression were observed between cancer patients and a control group matched for age and menopausal status (Favoni et al., 1995).

The IGF system has also been implicated in growth and progression of colon cancer. Anchorage-independent colony formation, a marker for progressive cellular transformation, is affected by the IGF-I pathway, because IGF-I receptor blocking antibodies severely inhibited colony formation in LS1034 colon cancer cells. The later stages of malignant progression in colorectal cancer cells are markedly influenced by IGFBP-4. Anchorage-independent colony formation was significantly reduced by IGFBP-4 via mechanisms independent of the functionality of the IGF/IGF receptor pathway and independent of IGF-II binding. In contrast, inhibitory activities of IGFBP-4 on cell proliferation and invasion of colon cancer cells also depend on its IGF-scavenging activities. (Diehl et al., 2004).

Insulin-like growth factors (IGFs) are important growth regulators of both normal and malignant prostate cells. IGFBP-4 immunostaining and hybridization signal were significantly increased in prostate adenocarcinoma compared to those in benign epithelium. IGFBP expression has been detected in a number of prostate cancer cell lines. (Tennant et al., 1996). In addition to PAPP-A, kallikrein 2 and kallikrein 3 (prostate specific antigen) can also cleave IGFBP4. Prostate specific antigen, PSA, levels are used as a serum marker of prostate cancer, both in diagnosis and monitoring response to therapy. The prostatic kallikreins hK2 and hK3 (prostate-specific antigen) may influence specifically the tumoral growth of prostate cells through the degradation of IGFBPS, to increase IGF bioavailability. hK3 cleaved IGFBP-4 but not IGFBP-2 and -5, whereas hK2 cleaved all of the IGFBPs much more effectively, and at concentrations far lower than those reported for other IGFBP-degrading proteases (Rehault et al., 2001).

In the M12 prostate cancer cell line, overexpression of IGFBP-4 was shown to delay tumorigenesis while decreasing the production of IGFBP-2. IGF-induced proliferation was reduced in the IGFBP-4 transfected cells compared with control cells. When injected s.c. into male athymic/nude mice, a marked delay was noted in tumor formation in animals receiving IGFBP-4 transfected cells (Damon et al., 1998). However, blocking IGFBP4 expression also inhibited tumour growth. Prostate cancer cell lines transfected with IGFBP4 antisense to block IGFBP4 expression, proliferated more slowly in monolayer culture than parental controls. Colony formation in soft agar was strongly inhibited and the rate of tumor formation and growth in male athymic nude mice injected with IGFBP4 antisense-transfected M12 cells was markedly reduced relative to that in mice receiving M12 control cells (Drivdahl R. H. et al., 2001).

Advanced prostate and breast cancers frequently involve the bone, which has the largest content of insulin-like growth factors (IGFs). Normal bone homeostasis is regulated by both systemic hormones and local growth factors, with insulin-like growth factors (IGFs) playing a pivotal role. (McCarthy T L et al., 1989; Mohan, S et al., 1991).

Although advances in the treatment of cancer have been made, there still exists a need for improved methods of treating cancer. In particular, a need remains for improved and effective therapies to treat cancer and metastases.

### SUMMARY OF THE INVENTION

According to the invention, there is provided a method of treating or preventing cancer in an individual in need thereof, comprising the step of administering to the individual a therapeutic amount of a modified IGF binding protein 4 (IGFBP4), or a polynucleic acid which encodes the modified IGFBP4 protein, wherein the protein is modified to be resistant to cleavage by pregnancy associated plasma protein A (PAPP-A).

In a preferred embodiment, the method is for therapy of individuals having established cancers. Typically, the cancer is selected from the group comprising: fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias. It is envisaged that the method of the invention may also be applicable for the treatment of other cancers.

The method of the invention may also be used prophylactically. Thus, in one embodiment, the method of the invention is a method of inhibiting or preventing the development of secondary tumours in individuals having established primary cancers. In a further embodiment, the method of the invention is a method of suppressing or preventing the development of metastases in individuals having established primary cancers. In a yet further embodiment of the invention, the method is a method of treating metastases, in particular bone metastases. Many other metastases may be prevented, or treated, by the methods of the invention, including lung and liver metastases.

Typically, the modified IGFBP4 protein is a recombinant mammalian protein, preferably a recombinant rat or mouse protein, and most preferably a recombinant human protein. Suitably, the modified IGFBP4 protein is a recombinant human protein having an amino acid sequence of SEQ ID NO:1.

The IGFBP4 protein is preferably made resistant to cleavage by PAPP-A by modifying the amino acid sequence of the protein. Typically, the protein is mutated to modify the amino acid sequence of the PAPP-A recognition domain of IGFBP4. The paper by Zhang et al. (2002) describes this domain as a 13 amino acid sequence stretching from residues 120 to 132 and having the amino acid sequence:

KHMAKVRDRSKMK (SEQ ID NO:3)

In one embodiment of the invention, this 13 residue domain is replaced by the sequence:

AAMAAVADASAMA (SEQ ID NO:4)

It will however be appreciated, the 13 residue binding domain may be modified in any other manner provided that the modified protein is rendered resistant to cleavage by the PAPP-A protease. Techniques for modifying the amino acid sequence of a protein (by either direct modification of the protein, or by indirect modification of a nucleic acid encoding the protein) by substitution, addition and/or deletion will be well known to these skilled in the art.

In one embodiment of the invention, the method includes administering a nucleic acid coding for the mutant protein to an individual. The nucleic acid may be administered directly (in vivo), either on its own or as part of a suitable vector, or indirectly, by administering cells previously transformed with the nucleic acid to the individual (ex vivo). Gene therapy techniques are discussed in more detail below.

The invention also relates to a method of inhibiting growth and proliferation of tumour cells in an individual in need thereof, comprising the step of administering to the individual a therapeutic amount of a modified IGF binding protein 4 (IGFBP4), or a nucleic acid which encodes the modified IGFBP4 protein, wherein the protein is modified to be resistant to cleavage by pregnancy associated plasma protein A (PAPP-A).

Typically, the tumour cell is selected from the group comprising: breast; prostrate; ovarian; and colon.

The invention also relates to a method of inhibiting the formation of metastases from primary tumours in an individual having an established primary tumour, comprising the step of administering to the individual a therapeutic amount of a modified IGF binding protein 4 (IGFBP4), or a nucleic acid which encodes the modified IGFBP4 protein, wherein the protein is modified to be resistant to cleavage by pregnancy associated plasma protein A (PAPP-A).

Typically, the established primary tumour is selected from the group comprising: breast; prostrate; and ovarian.

The invention also relates to a composition, suitably a pharmaceutical composition, for preventing or treating cancer comprising a therapeutic amount of a modified IGF binding protein 4 (IGFBP4) and a physiologically acceptable carrier or excipient, wherein the protein is modified to be resistant to cleavage by pregnancy associated plasma protein A (PAPP-A).

The invention also relates to a composition, suitably a pharmaceutical composition, for treating cancer comprising: a polynucleotide encoding a modified IGF binding protein 4 (IGFBP4); and a physiologically acceptable carrier or excipient, wherein the protein is modified to be resistant to cleavage by pregnancy associated plasma protein A (PAPP-A). Typically, the polynucleotide is contained within an expression vector.

The invention also relates to a composition, suitably a pharmaceutical composition, for treating cancer comprising: cells transformed with a polynucleotide encoding a modified IGF binding protein 4 (IGFBP4); and a physiologically acceptable carrier or excipient, wherein the protein is modified to be resistant to cleavage by pregnancy associated plasma protein A (PAPP-A).

These and other embodiments of the invention will be described in further detail in connection with the detailed description of the invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a schematic drawing of the effects of IGFBP4 binding to IGF I. FIG. 1A: IGF I (or IGF II) bound to IGFBP4 is released when PAPP-A protease cleaves IGFBP4. IGF can then stimulate cell proliferation and angiogenesis. FIG. 1B: When IGF is bound to protease-resistant IGFBP4, PAPP-A cannot cleave IGFBP4. IGF remains bound to IGFBP4 and is therefore inactive.

FIG. 2 shows IGFBP4 (intact and cleavage fragments) and PAPP-A expression in experimental mouse lung and bone metastases compared to normal lung and normal bone tissue.

FIG. 3 shows 4T1.2 cell proliferation in response to IGF-1 and IGF-1(RE), which is resistant to IGFBP4 binding. IGF-1 that is not bound by IGFBP4 stimulates cell proliferation whereas IGF-1 bound by IGFBP4 does not. (*p<0.05 vs control).

FIG. 4 shows that IGF-1(RE) stimulates VEGF production by 4T1.2 cells.

FIG. 5 shows that overexpression of protease-resistant IGFBP4 increases survival in tumour-bearing mice. 4T1.2 tumour cells transfected with control plasmid (pCMV), plasmid expressing wild type IGFBP4 (BP4) or plasmid expressing protease-resistant IGFBP4 (ΔBP4) were implanted in the mammary fat pad of BALB/c mice (n=10/group). Tumours were allowed to grow to mean tumour diameter of 17 mm, at which time mice were sacrificed. Data representative of two independent experiments are presented.

FIG. 6 shows increased endothelial cell apoptosis in tumours expressing protease resistant IGFBP4. 4T1.2 tumours transfected with control plasmid (pCMV), plasmid expressing wild type IGFBP4 (BP4) or plasmid expressing protease-resistant IGFBP4 (ΔBP4) were stained with CD-31 (red) to identify blood vessels, TUNEL (green) to identify apoptotic cells and DAPI (blue) to highlight nuclei stained with DAPI. 400× magnification. Panels on right hand side were stained with CD31 using DAB chromogen.

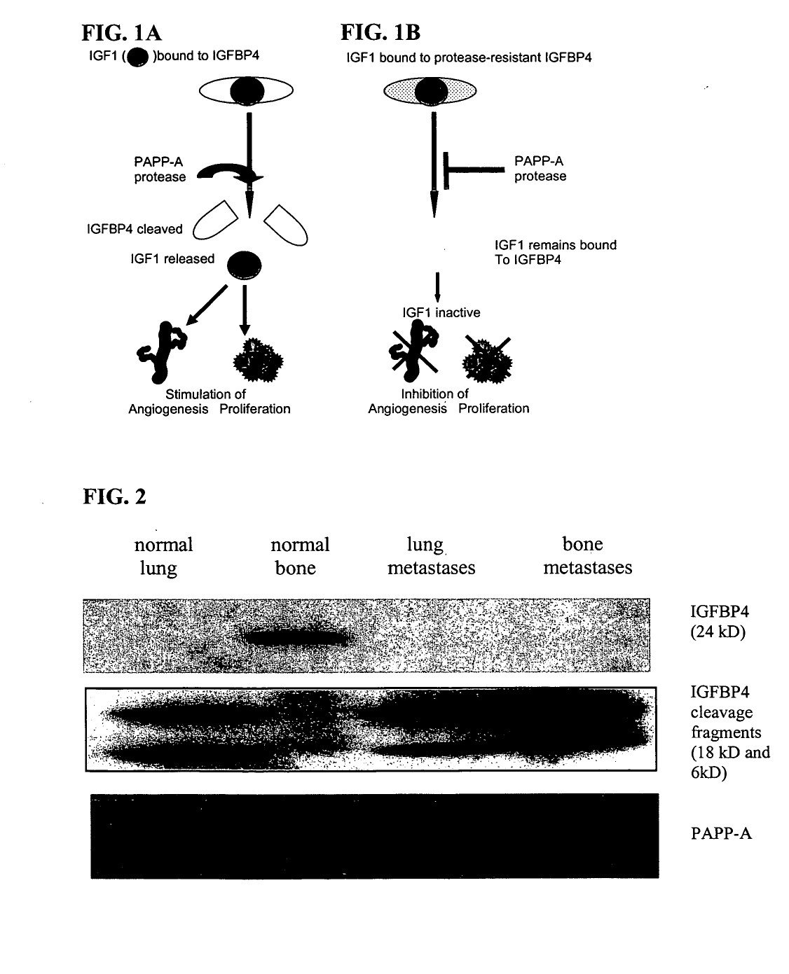

### DETAILED DESCRIPTION OF THE INVENTION

Protease Resistant IGFBP4

A protease-resistant clone of rat IGFBP4 was obtained from James Fagin (University of Cincinnati). The production of PAPP-A resistant IGFBP4 cDNA (ΔBP4), and the rat recombinant IGFBP4 mutant, is described in detail in the Experimental Procedures section of Zhang et al. (2002), the full reference of which is included in the References section below, and the full content of which is incorporated herein by reference in its entirety. In essence, the cDNA was generated by changing the DNA sequence at the PAPP-A cleavage sites within IGFBP4. The altered DNA sequence results in amino acid changes at the cleavage site, such that the protein is resistant to cleavage by PAPP-A but retains its ability to bind IGFI or IGFII.

The general methods employed to generate recombinant mutant IGFBP4 are as follows. The protease resistant IGFBP4 DNA sequence (ΔBP4) is cloned into a plasmid expression vector containing a histidine or glutathione S transferase (GST) tag to facilitate purification, and under the control of a strong constitutive promoter such that the protein is expressed at very high levels. The vector containing the protease resistant IGFBP4 DNA sequence (pΔBP4) is then introduced into either bacterial or mammalian cells. The histidine tagged ABP4 protein is expressed and secreted by the transformed cells. The histidine (HIS) tag consists of a string of histidine amino acid residues at either the 3′ or 5′ end of the protein (more usually the 5′ end). Culture medium, containing the HIS-tagged ΔBP4 protein, is then passed through a nickel affinity column which binds the HIS tag (or glutathione agarose if GST tagged). The HIS tagged ΔBP4 protein is then eluted from the column. The HISD/GST tag may or may not be removed. Depending on the purity of the recovered protein, additional purification steps may be employed. The purified protein is then assayed for IGF1 binding capacity and resistance to PAPP-A cleavage.

The amino acid sequence of rat IGFBP4 protein (Accession number NP_{—}001004274; SEQ ID NO:5) is presented below:

| | |
|---|---|
| 1 | MLPFGLVAAL LLAAGPRPSL GDEAIHCPPC SEEKLARCRP PVGCEELVRE PGCGCCATGA |
| 61 | LGLGMPCGVY TPRCGSGMRC YPPRGVEKPL RTLMHGQGVC TELSEIEAIQ ESLQTSDKDE |
| 121 | SEHPNNSFNP CSAHDHRCLQ KHMAKVRDRS KMKVVGTPRE EPRPVPQGSC QSELHRALER |
| 181 | LAASQSRTHE DLFIIPIPNC DRNGNFHPKQ CHPALDGQRG KCWCVDRKTG VKLPGGLEPK |
| 241 | GELDCHQLAD SLQE |

The underlined region is mutated in protease-resistant rat IGFBP4 (SEQ ID NO:6) to AAMAAVADASAMA (SEQ ID NO:4).

The numbering of the underlined cleavage site differs from the numbering of the cleavage site in Zhang et al. (2002) due to the fact that the above sequence includes a pre-sequence which is cleaved off after the protein is secreted.

The amino acid sequence of human IGFBP4 protein (Accession number AAH 16041; SEQ ID NO:7) is presented below:

| | |
|---|---|
| 1 | MLPLCLVAAL LLAAGPGPSL GDEAIHCPPC SEEKLARCRP PVGCEELVRE PGCGCCATCA |
| 61 | LGLGMPCGVY TPRCGSGLRC YPPRGVEKPL HTLMHGQGVC MELAEIEAIQ ESLQPSDKDE |
| 121 | GDHPNNSFSP CSAHDRRCLQ KHFAKIRDRS TSGGKMKVNG APREDARPVP QGSCQSELHR |
| 181 | ALERLAASQS RTHEDLYIIP IPNCDRNGNF HPKQCHPALD GQRGKCWCVD RKTGVKLPGG |
| 241 | LEPKGELDCH QLADSFRE |

The underlined region is mutated in mutant human IGFBP4 to AAMAAVADASTSGGAMA (SEQ ID NO:8).

The amino acid sequence of mutant human IGFBP4, including the underlined altered region, is provided in SEQ ID NO:1.

The amino acid sequence of mouse IGFBP4 protein (Accession number AAH19836; SEQ ID NO:9) is presented below:

| | |
|---|---|
| 1 | MLPFGLVAAL LLAAGPRPSL GDEAIHCPPC SEEKLARCRP PVGCEELVRE PGCGCCATCA |
| 61 | LGLGMPCGVY TPRCGSGMRC YPPRGVEKPL RTLMHGQGVC TELSEIEAIQ ESLQTSDKDE |
| 121 | SEHPNNSFNP CSAHDHRCLQ KHMAKIRDRS KMKIVGTPRE EPRPVPQGSC QSELHRALER |
| 181 | LAASQSRTHE DLFIIPIPNC DRNGNFHPKQ CHPALDGQRG KCWCVDRKTG VKLPGGLEPK |
| 241 | GELDCHQLAD SFQE |

The underlined region is mutated in mutant mouse IGFBP4 to AAMAAVADASAMA (SEQ ID NO:4).

The amino acid sequence of mutant mouse IGFBP4, including the underlined altered region, is provided in SEQ ID NO:2.

Experimental

The Applicant has established that 4T1.2 mouse mammary adenocarcinoma growth and production of the angiogenic protein VEGF (vascular endothelial growth factor) are stimulated by IGF1. Although 4TI.2 tumour cells express high levels of IGFBP4, host cells within these tumours express high levels of the PAPP-A protease, which we have shown results in cleavage of IGFBP4 within these tumours. 4T1.2 tumours growing in bone contain particularly high levels of PAPP-A and consequently high levels of IGFBP4 cleavage fragments but no intact IGFBP4 (FIG. 2). In both tumours and normal tissues, PAPP-A and IGFBP4 are expressed in an inverse pattern—in tissues with high levels of PAPP-A, there is little or no intact IGFBP4 and tissues expressing high levels of IGFBP4 have low levels of PAPP-A.

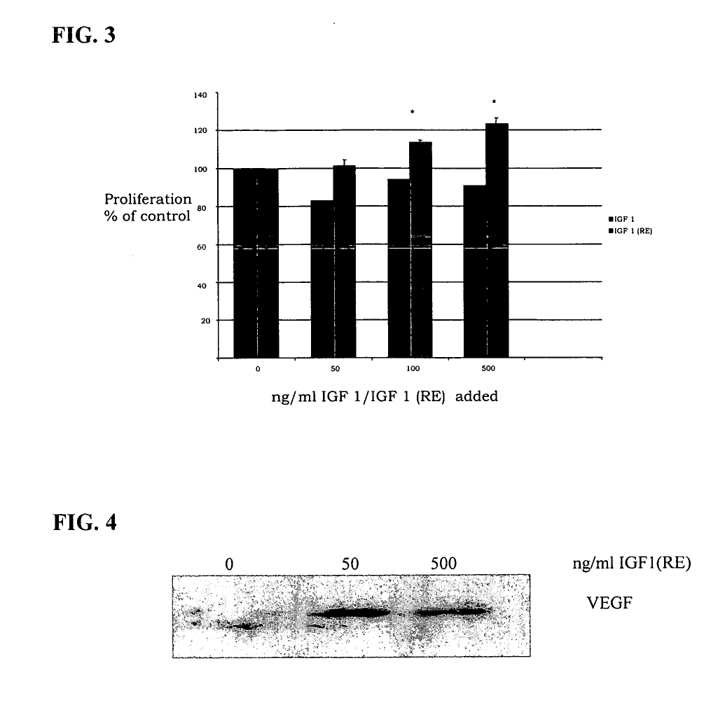

The Applicant has also shown, in vitro, that in the absence of PAPP-A, 4T1.2 cells produce high levels of IGFBP4 which blocks IGF1 stimulation of tumour cell proliferation and VEGF production. Cells were treated with either IGF1 or a mutant IGF1 (IGF1RE), which is resistant to IGFBP4 binding. The mutant IGFBP4 resistant IGF1 that is not bound by IGFBP4 stimulates cell proliferation whereas wild type IGF 1 bound by IGFBP4 does not (FIG. 3). Similarly, IGF1RE stimulates production of the angiogenic factor, vascular endothelial growth factor, VEGF (FIG. 4).

Administration of ΔBP4 protein to tumour bearing mice should result in IGF 1 binding to the ΔBP4 protein. As the ΔBP4 protein cannot be cleaved by PAPP-A, IGF1 binding to the ΔBP4 protein will prevent IGF1 stimulation of tumour cell proliferation and VEGF production, thereby inhibiting tumour growth and metastasis.

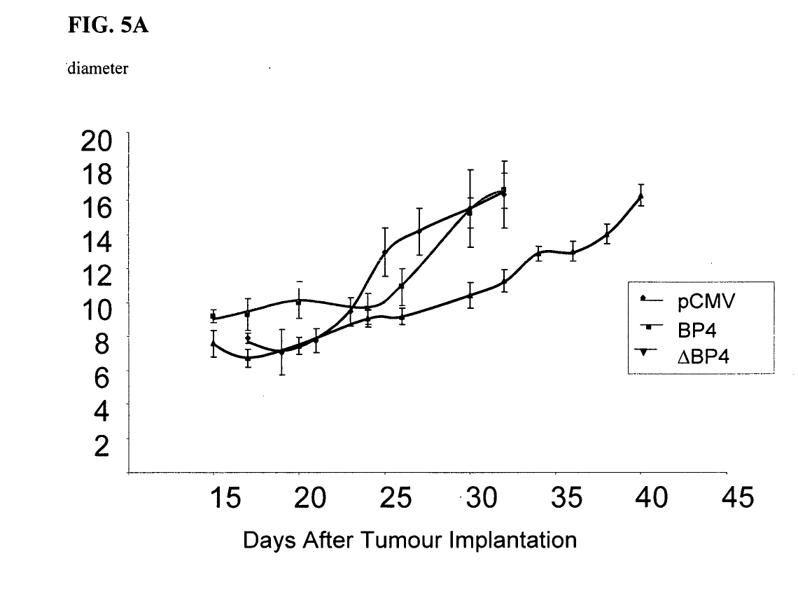

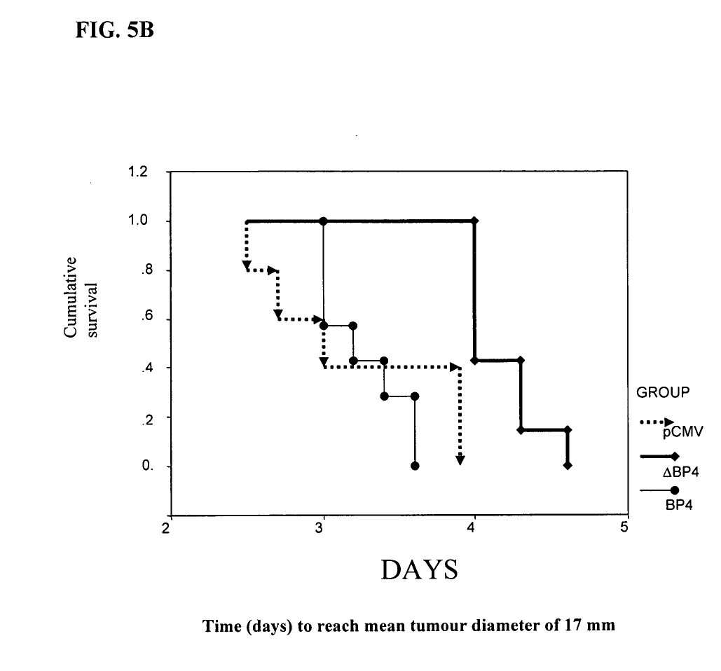

To establish the antitumour efficacy of ΔBP4 protein, 4T1.2 mammary adenocarcinoma cells were transfected with control plasmid (pCMV), plasmid expressing wild type IGFBP4 (BP4), or plasmid expressing protease-resistant IGFBP4 (ΔBP4) using techniques well known in the art. These transfected cells were implanted in the mammary fat pad of BALB/c mice (n=10/group) and the growth of tumours monitored by measuring mean tumour diameter every second day. ΔBP4-transfected 4T1.2 cells grew slower than control-transfected cells resulting in a statistically significant survival advantage (expressed as time to reach mean tumour diameter of 17 mm) (FIGS. 5A and 5B). Data were analysed using Stata Release 8.2 (StataCorp LP, College Station, Tex.). Examination of the data showed that the survival curves for control and BP4 were similar, and a logrank test revealed no difference between the two groups (P=0.598). Compared with the control and BP4 groups, however, the ΔBP4 group had an extended survival (Chi-sq=16.4, p<0.0001).

In addition, 4T1.2 cells implanted in the mammary fat pad are capable of forming spontaneous lung and bone metastases. Serum bone markers suggestive of presence of bone metastases (calcium, phosphate and alkaline phosphatase) were measured from mice implanted with 4T1.2 cells transfected with pCMV, BP4 or ΔBP4 plasmids (Table 1).

TABLE 1
Serum bone markers

| | Ca | PO₄ | Alk Phos |
|---|---|---|---|
| pCMV | 2.502 | 2.7 | 67.25 |
| BP4 | 2.66 | 3.047 | 79.00 |
| ΔBP4 | 2.442 | 2.244 | 55.6 |

Calcium, phosphate and alkaline phosphatase were reduced in mice implanted with ΔBP4-transfected 4T1.2 cells relative to the control pCMV transfected cells, suggesting that bone metastases were reduced.

This data clearly demonstrates that ΔBP4 will inhibit tumour growth.

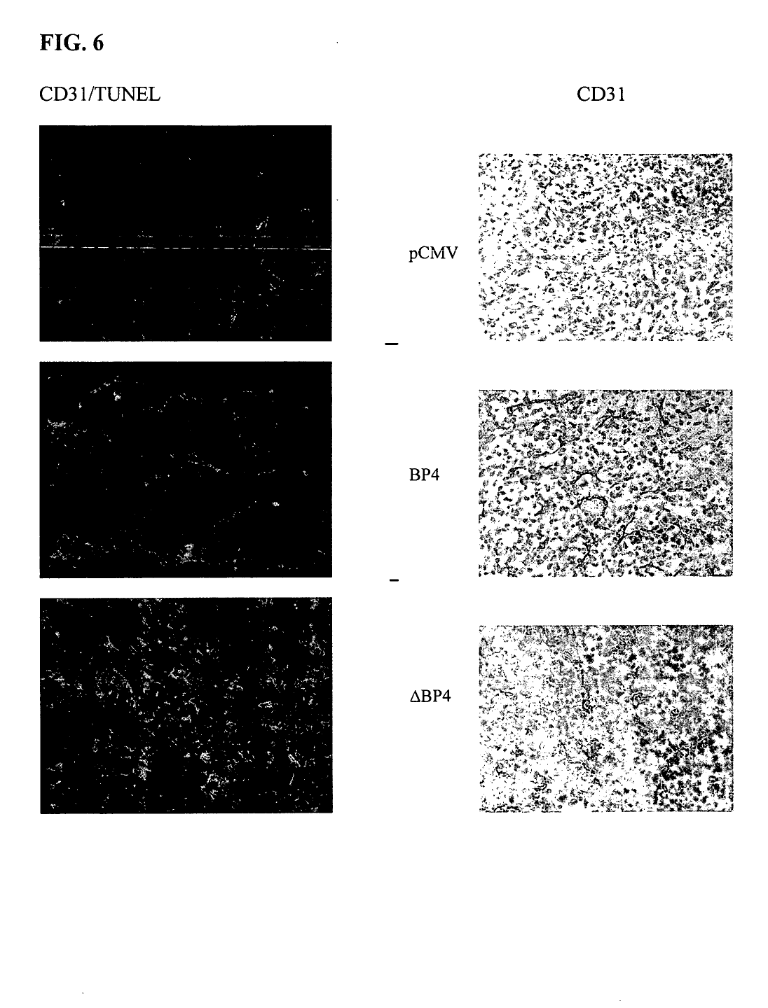

4T1.2 tumours transfected with control plasmid (pCMV), plasmid expressing wild type IGFBP4 (BP4) or plasmid expressing protease-resistant IGFBP4 (ΔBP4) were stained with CD-31 (red) to identify blood vessels, TUNEL (green) to identify apoptotic cells and DAPI (blue) to highlight nuclei stained (FIG. 6).

Double staining of tumour sections with CD31 to identify blood vessels and TUNEL to identify apoptosis identified increased numbers of apoptotic endothelial cells in tumours expressing protease resistant IGFBP4 (6.97+/−3.26 apoptotic endothelial cells/high power field) with little or no endothelial cell apoptosis in control tumours (0.90+/−0.50 apoptotic endothelial cells/high power field) or tumours expressing wild type IGFBP4 (1.20+/−0.95 apoptotic endothelial cells/high power field). In addition, CD31 immunohistochemistry demonstrates that the vessels in tumours expressing protease resistant IGFBP4 have altered morphology with discontinuous endothelium and occluded lumens visible in contrast to clear lumens in control tumours and those expressing wild type IGFBP4. These data are consistent with an anti-angiogenic mechanism underlying inhibition of tumour growth in response to protease resistant IGFBP4 expression.

Homology of Rat, Mouse and Human IGFBP4

Using the BLAST alignment program (available from the website of the National Center for Biotechnology Information: ncbi.nlm.nih.gov), rat (SEQ ID NO: 5) and human (SEQ ID NO: 7) IGFBP4 sequences were aligned and found to be 85% homologous (see below); mouse (SEQ ID NO:9) and human (SEQ ID NO:7) IGFBP4 sequences also are 85% homologous.

| | | | |
|---|---|---|---|
| Query (rat): | 22 | DEAIHCPPCSEEKLARCRPPVGCEELVREPGCGCCATCALGLGMPCGVYTPRCGSGMRCY | 81 |
| Sbjct (human): | 22 | DEAIHCPPCSEEKLARCRPPVGCEELVREPGCGCCATCALGLGMPCGVYTPRCGSGLRCY | 81 |
| Query (rat): | 82 | PPRGVEKPLRTLMHGQGVCTELSEIEAIQESLQTSDKDESEHPNNSFNPCSAHDHRCLQK | 141 |
| Sbjct (human): | 82 | PPRGVEKPLHTLMHGQGVCMELAEIEAIQESLQPSDKDEGDHPNNSFSPCSAHDRRCLQK | 141 |
| Query (rat): | 142 | HMAKVRDRS----KMKVVGTPREEPRPVPQGSCQSELHRALERLAASQSRTHEDLFIIPI | 197 |
| Sbjct (human): | 142 | HFAKIRDRSTSGGKMKVNGAPREDARPVPQGSCQSELHRALERLAASQSRTHEDLYIIPI | 201 |
| Query (rat): | 198 | PNCDRNGNFHPKQCHPALDGQRGKCWCVDRKTGVKLPGGLEPKGELDCHQLADSLQE | 254 |
| Sbjct (human): | 202 | PNCDRNGNFHPKQCHPALDGQRGKCWCVDRKTGVKLPGGLEPKGELDCHQLADSFRE | 258 |

Gene Therapy

In a specific embodiment, nucleic acids comprising a sequence encoding a PAPP-A protease resistant IGFBP4 are administered for treatment or prevention of cancer by way of gene therapy. Gene therapy refers to therapy performed by the administration of a nucleic acid to a subject. In this embodiment of the invention, the nucleic acid produces its encoded protein that mediates a therapeutic effect. For example, any of the methods for gene therapy available in the art can be used according to the present invention. Examples of such methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the nucleic acid encoding the PAPP-A resistant IGFBP4 is part of an expression vector that produces the recombinant protein in a suitable host. In particular, such a nucleic acid has a promoter operably linked to the nucleic acid sequence coding for the recombinant protein, said promoter being inducible or constitutive, and, optionally, tissue-specific. In another particular embodiment, a nucleic acid molecule is used in which the mutant IGFBP4 sequences and any other desired sequences are flanked by regions that promote homologous recombination at a desired site in the genome, thus providing for intrachromosomal expression of the mutant protein (Koller and Smithies, 1989, Proc. Natl. Acad. Sci. USA 86:8932-8935; Zijistra et al. 1989, Nature 342:435-438).

Delivery of the nucleic acid into a patient maybe either direct, in which case the patient is exposed directly to the nucleic acid or nucleic acid-carrying vector, or indirect, in which case, cells are transformed with the nucleic acid in vitro first, then administered to the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the cell or nucleus, e.g., by administering it in linkage to a ligand subject to receptor-mediated endocytosis (see e.g., Wu and Wu, 1987, J. Biol. Chem. 262:4429-4432) (which can be used to target cell types specifically expressing the receptors), etc. In a specific embodiment, the nucleic acid can be targeted in vivo for cell specific uptake and expression, by targeting a specific receptor (see for example PCT Publications WO92/20316; WO93/14188; and WO93/20221.

In a specific embodiment, a viral vector that contains the nucleic acid sequence encoding the mutant IGFBP4 may be employed. For example, a retroviral vector can be used (see Miller et al., 1993, Meth. Enzymol. 217:581-599). These retroviral vectors have been modified to delete retroviral sequences that are not necessary for packaging of the viral genome. Retroviral vectors are maintained in infected cells by integration into genomic sites upon cell division. The nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review, of adenovirus-based gene therapy. Bout et al., 1994, Human Gene Therapy 5:3-10 demonstrated the use of adenovirus vectors to transfer genes to the respiratory epithelia of rhesus monkeys.

Adeno-associated virus (AAV) has also been proposed for use in gene therapy (Walsh et al., 1993, Proc. Soc. Exp. Biol. Med. 204:289-300). Herpes viruses are other viruses that can also be used.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including, but not limited to, transfection, electroporation, microinjection, infection with a viral vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, recombinant blood cells (e.g., hematopoietic stem or progenitor cells) are administered intravenously. Additionally, epithelial cells can be injected, e.g., subcutaneously, or recombinant skin cells (e.g., keratinocytes) may be applied as a skin graft onto the patient. The amount of cells envisioned for use depends on the desired effect, patient state, etc., and can be determined by one skilled in the art.

In an embodiment in which recombinant cells are used in gene therapy, a nucleic acid sequence coding for the mutant IGFBP4 is introduced into the cells such that it is expressible by the cells or their progeny, and the recombinant cells are then administered in vivo for therapeutic effect. In a specific embodiment, stem or progenitor cells, preferably hematopoietic stem or progenitor cells, are used. Any stem and/or progenitor cells which can be isolated and maintained in vitro can potentially be used in accordance with this embodiment of the present invention.

Many methods of gene therapy are available in the art (for general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the nucleic acid which provides a gene product desired in a subject is introduced into an expression vector that produces the gene product. In particular, such a nucleic acid has a promoter operably linked to the nucleic acid sequence of interest, said promoter being inducible or constitutive, and, optionally, tissue-specific. In another particular embodiment, a nucleic acid molecule is used in which the sequences of interest are flanked by regions that promote homologous recombination at a desired site in the genome, thus providing for intrachromosomal expression of the desired protein (Koller and Smithies, 1989, Proc. Natl. Acad. Sci. USA 86:8932-8935; Zijlstra et al., 1989, Nature 342:435-438).

Therapeutic Compositions and Methods of Administration

The invention provides methods of, and compositions for, treatment and prevention by administration to a subject in need of such treatment of a therapeutically or prophylactically effective amount of a therapeutic of the invention. The subject may be an animal or a human, with or without an established cancer.

Various delivery systems are known and can be used to administer a therapeutic of the invention, e.g., encapsulation in liposomes, microparticles, microcapsules, recombinant cells capable of expressing the therapeutic, receptor-mediated endocytosis (see, e.g., Wu and Wu, 1987, J. Biol. Chem. 262:4429-4432), construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the compositions of the invention locally to the area in need of treatment; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

Alternatively, the therapeutic can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327.)

In yet another embodiment, the therapeutic can be delivered in a controlled release system. In one embodiment, a pump may be used (see Langer, supra; Sefton, CRC Crit. Ref. Biomed., Eng. 14:201 (1987); Buchwald et al., Surgery 88:75 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

The present invention also provides pharmaceutical compositions. Such compositions comprise a therapeutically effective amount of a therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the therapeutic of the invention which will be effective in the treatment or prevention of cancer will depend on the type, stage and locus of the cancer, and, in cases where the subject does not have an established cancer, will depend on various other factors including the age, sex, weight, and clinical history of the subject. The amount of therapeutic may be determined by standard clinical techniques. In addition, in vivo and/or in vitro assays may optionally be employed to help predict optimal dosage ranges. The precise dose to be employed in the formulation will also depend on the route of administration, and the seriousness of the cancer, and should be decided according to the judgment of the practitioner and each patient's circumstances. Routes of administration of a therapeutic include, but are not limited to, intramuscularly, subcutaneously or intravenously. Effective doses may be extrapolated from dose-response curves derived from in vitro or animal model test systems.

The invention also provides a pharmaceutical pack or kit comprising one or more containers filled with one or more of the ingredients of the compositions of the invention.

The invention is not limited to the embodiments hereinbefore described which may be varied in both construction and detail without departing from the spirit of the invention. In particular, the invention is not limited to the use of the specific protease resistant proteins disclosed herein.

### REFERENCES

Rasmussen A A, Cullen K J. Paracrine/autocrine regulation of breast cancer by the insulin-like growth factors. Breast Cancer Res Treat 1998, 47: 219-33.
Peyrat J P, Bonneterre J, Hecquet B, Vennin P, Luuchez M M, Fournier C, Lefebvre J, Demaille A. Plasma insulin-like growth factor-I (IGF-I) concentrations in human breast cancer. Eur J Cancer 1993, 29: 492-7.
Kucab J E, Dunn S E. Role of IGF-1 R in Mediating Breast Cancer Invasion and Metastasis. Breast Dis. 2003;17:41-7.
Ng E H, Ji C Y, Tan P H, Lin V, Soo K C, Lee K O. Altered serum levels of insulin-like growth-factor binding proteins in breast cancer patients. Ann Surg Oncol. 1998 March;5(2):194-201.
Yee D, Sharma J, Hilsenbeck S G. Prognostic significance of insulin-like growth factor binding protein expression in axillary lymph node negative breast cancer. J Natl Cancer Inst 1994, 86: 1785-9.
Favoni R E, de Cupis A, Perrotta A, Sforzini S, Amoroso, Pensa F, Miglietta L. Insulin-like growth factor-I (IGF-I) and IGF-binding proteins blood serum levels in women with early- and late-stage breast cancer: mutual relationship and possible correlations with patients' hormonal status. J Cancer Res Clin Oncol. 1995;121(11):674-82.
Diehl D, Hoeflich A, Wolf E, Lahm H. Insulin-like growth factor (IGF)-binding protein-4 inhibits colony formation of colorectal cancer cells by IGF-independent mechanisms. Cancer Res. 2004 Mar. 1;64(5):1600-3.
Tennant M K, Thrasher J B, Twomey P A, Birnbaum R S, Plymate S R. Insulin-like growth factor-binding proteins (IGFBP)-4, -5, and -6 in the benign and malignant human prostate: IGFBP-5 messenger ribonucleic acid localization differs from IGFBP-5 protein localization. J Clin Endocrinol Metab. 1996 October;81(10):3783-92.
Rehault S, Monget P, Mazerbourg S, Tremblay R, Gutman N, Gauthier F, Moreau T. Insulin-like growth factor binding proteins (IGFBPs) as potential physiological substrates for human kallikreins hK2 and hK3. Eur J Biochem. 2001 May;268(10):2960-8.
Damon S E, Maddison L, Ware J L, Plymate S R. Overexpression of an inhibitory insulin-like growth factor binding protein (IGFBP), IGFBP-4, delays onset of prostate tumor formation. Endocrinology. 1998 August;139(8):3456-64.
Drivdahl R H, Sprenger C, Trimm K, Plymate S R. Inhibition of growth and increased expression of insulin-like growth factor-binding protein-3 (IGFBP-3) and -6 in prostate cancer cells stably transfected with antisense IGFBP-4 complementary deoxyribonucleic acid. Endocrinology. 2001 May;142(5):1990-8.
McCarthy T L, Centrella M, Canalis E. Insulin-like growth factor (IGF) and bone. Connective Tissue Res 1989, 20:277-82.
Mohan S, Baylink D J. Bone growth factors. Clin Orthop 1991, 26.
Zhang M, Smith E, Kuroda H, Banach W, Chemausek S, Fagin J. Targeted expression of a protease resistant IGFBP-4 mutant in smooth muscle of transgenic mice results in IGFBP-4 stabilisation and smooth muscle hypotrophy. J. Biol. Chem. 2002 June; 277(24):21285-21290
   Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method of treating or preventing cancer in an individual in need thereof, comprising the step of administering to the individual a therapeutic amount of a modified IGF binding protein 4 (IGFBP4), or a nucleic acid which encodes the modified IGFBP4 protein, wherein the protein is modified to be resistant to cleavage by pregnancy associated plasma protein A (PAPP-A).

2. The method claimed in claim 1, in which the modified IGFBP4 protein is a recombinant mammalian protein.

3. The method claimed in claim 1, in which the modified IGFBP4 protein is a recombinant rat protein.

4. The method claimed in claim 1, in which the modified IGFBP4 protein is a recombinant human protein.

5. The method claimed in claim 1, in which the modified IGFBP4 protein is a recombinant human protein having an amino acid sequence set forth as SEQ ID NO: 1or SEQ ID NO:2.

6. The method claimed in claim 1, in which the IGFBP4 protein is modified by changing the amino acid sequence at the PAPP-A cleavage site.

7. The method claimed in claim 1, in which the cancer is a primary tumour.

8. The method claimed in claim 1, in which the cancer is a metastasis.

9. The method claimed in claim 8, in which the metastasis is selected from the group comprising: bone metastases; lung metastases; and liver metastases.

10. The method claimed in claim 1, in which the cancer is selected from the group comprising: fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

11. The method claimed in claim 1, in which the individual is treated with an expression vector comprising the nucleic acid encoding the modified protein.

12. A method of inhibiting growth and proliferation of tumour cells in an individual in need thereof, comprising the step of administering to the individual a therapeutic amount of a modified IGF binding protein 4 (IGFBP4), or a nucleic acid which encodes the modified IGFBP4 protein, wherein the protein is modified to be resistant to cleavage by pregnancy associated plasma protein A (PAPP-A).

13. The method claimed in claim 12, in which the tumour cell is selected from the group comprising: breast; prostrate; and ovarian.

14. A method of inhibiting the formation of metastases from primary tumours in an individual having an established primary tumour, comprising the step of administering to the individual a therapeutic amount of a modified IGF binding protein 4 (IGFBP4), or a nucleic acid which encodes the modified IGFBP4 protein, wherein the protein is modified to be resistant to cleavage by pregnancy associated plasma protein A (PAPP-A).

15. The method claimed in claim 14, in which the established primary tumour is selected from the group comprising: breast; prostrate; ovarian; and colon.

16. The method claimed in claim 14, in which the metastases are selected from the group comprising: bone; lung; and liver.

17. A method of inhibiting the growth of metastases in an individual, comprising the step of administering to the individual a therapeutic amount of a modified IGF binding protein 4 (IGFBP4), or a nucleic acid which encodes the modified IGFBP4 protein, wherein the protein is modified to be resistant to cleavage by pregnancy associated plasma protein A (PAPP-A).

18. The method claimed in claim 17, in which the metastases is selected from the group comprising: bone; lung; and liver.

19. A composition for treating cancer comprising a therapeutic amount of a modified IGF binding protein 4 (IGFBP4) and a physiologically acceptable carrier or excipient, wherein the protein is modified to be resistant to cleavage by pregnancy associated plasma protein A (PAPP-A).

20. A composition for treating cancer comprising: a polynucleotide encoding a modified IGF binding protein 4 (IGFBP4); and a physiologically acceptable carrier or excipient, wherein the protein is modified to be resistant to cleavage by pregnancy associated plasma protein A (PAPP-A).

21. The composition claimed in claim 20, in which the polynucleotide is contained within an expression vector.


SEQUENCE LISTING (9 sequences)

SEQ ID NO: 1 - protein, 258 aa, Homo sapiens
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQAAMAAVADASTSGGAMAVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 2 - protein, 254 aa, Mus musculus
MLPFGLVAALLLAAGPRPSLGDEAIHCPPCSEEKLARCRPPVGCEELVREPGCGCCATCALGLGMPCGVYTPRCGSGMRCYPPRGVEKPLRTLMHGQGVCTELSEIEAIQESLQTSDKDESEHPNNSFNPCSAHDHRCLQAAMAAVADASAMAIVGTPREEPRPVPQGSCQSELHRALERLAASQSRTHEDLFIIPIPNCDRNGNFHPKQCHPALDGQRGKCWCVDRKTGVKLPGGLEPKGELDCHQLADSFQE

SEQ ID NO: 3 - protein, 13 aa, Rattus norvegicus
KHMAKVRDRSKMK

SEQ ID NO: 4 - protein, 13 aa, Artificial
  mutant sequence
AAMAAVADASAMA

SEQ ID NO: 5 - protein, 254 aa, Rattus norvegicus
MLPFGLVAALLLAAGPRPSLGDEAIHCPPCSEEKLARCRPPVGCEELVREPGCGCCATCALGLGMPCGVYTPRCGSGMRCYPPRGVEKPLRTLMHGQGVCTELSEIEAIQESLQTSDKDESEHPNNSFNPCSAHDHRCLQKHMAKVRDRSKMKVVGTPREEPRPVPQGSCQSELHRALERLAASQSRTHEDLFIIPIPNCDRNGNFHPKQCHPALDGQRGKCWCVDRKTGVKLPGGLEPKGELDCHQLADSLQE

SEQ ID NO: 6 - protein, 254 aa, Rattus norvegicus
MLPFGLVAALLLAAGPRPSLGDEAIHCPPCSEEKLARCRPPVGCEELVREPGCGCCATCALGLGMPCGVYTPRCGSGMRCYPPRGVEKPLRTLMHGQGVCTELSEIEAIQESLQTSDKDESEHPNNSFNPCSAHDHRCLQAAMAAVADASAMAVVGTPREEPRPVPQGSCQSELHRALERLAASQSRTHEDLFIIPIPNCDRNGNFHPKQCHPALDGQRGKCWCVDRKTGVKLPGGLEPKGELDCHQLADSLQE

SEQ ID NO: 7 - protein, 258 aa, Homo sapiens
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 8 - protein, 17 aa, Artificial
  mutant sequence
AAMAAVADASTSGGAMA

SEQ ID NO: 9 - protein, 254 aa, Mus musculus
MLPFGLVAALLLAAGPRPSLGDEAIHCPPCSEEKLARCRPPVGCEELVREPGCGCCATCALGLGMPCGVYTPRCGSGMRCYPPRGVEKPLRTLMHGQGVCTELSEIEAIQESLQTSDKDESEHPNNSFNPCSAHDHRCLQKHMAKIRDRSKMKIVGTPREEPRPVPQGSCQSELHRALERLAASQSRTHEDLFIIPIPNCDRNGNFHPKQCHPALDGQRGKCWCVDRKTGVKLPGGLEPKGELDCHQLADSFQE